(19) United States
(12) Patent Application Publication
(10) Pub. No.: US 2022/0354407 A1
(43) Pub. Date: Nov. 10, 2022

(54) RECONFIGURABLE ANALOG FRONT END FOR BIOSIGNAL ACQUISITION

(71) Applicant: RENSSELAER POLYTECHNIC INSTITUTE, Troy, NY (US)

(72) Inventors: Yu-Pin Hsu, Santa Clara, CA (US); Zemin Liu, Santa Clara, CA (US); Mona Mostafa Hella, Watervliet, NY (US)

(73) Assignee: RENSSELAER POLYTECHNIC INSTITUTE, Troy, NY (US)

(21) Appl. No.: 17/293,241

(22) PCT Filed: Nov. 13, 2019
(86) PCT No.: PCT/US19/61122

§ 371 Date: May 12, 2021

Related U.S. Application Data
(60) Provisional application No. 62/934,496, filed on Nov. 12, 2019.
(60) Provisional application No. 62/760,653, filed on Nov. 13, 2018.

(51) Int. Cl. A61B 5/301; A61B 5/304; A61B 5/308; A61B 5/311; A61B 5/00

(52) U.S. Cl. CPC A61B 5/301; A61B 5/304; A61B 5/308; A61B 5/311; A61B 5/7225

(57) ABSTRACT

In an embodiment, there is provided an apparatus. The apparatus includes an analog front end for biosignal acquisition. The analog front end includes an instrumentation amplifier and a reconfigurable filter. The instrumentation amplifier is configured to receive a biosignal and includes a super class-AB output stage. The reconfigurable filter is coupled to an output of the instrumentation amplifier. The reconfigurable filter has a selectable gain and an adjustable bandwidth. The bandwidth is adjusted based, at least in part, on a duty cycle of a clock signal.

### CROSS REFERENCE TO RELATED APPLICATION(S)

This application claims the benefit of U.S. Provisional Application No. 62/760,653, filed Nov. 13, 2018, and U.S. Provisional Application No. 62/934,496, filed Nov. 12, 2019, which are incorporated by reference as if disclosed herein in their entireties.

### GOVERNMENT LICENSE RIGHTS

This invention was made with government support under award number N68335-16-C-0117, awarded by the Office of Naval Research. The government has certain rights in the invention.

### FIELD

The present disclosure relates to an analog front end, in particular to, a reconfigurable analog front end for biosignal applications.

### BACKGROUND

Advances in wearable health monitoring technology have spurred a growing demand for sensing and acquisition of multiple physiological biosignals. The biosignals may then facilitate providing a complete health status evaluation. An important aspect of such trend is a reduction in energy consumption and device form factor to prolong battery lifetime and allow for wearable/implantable devices. Common biosignals, including, for example, Electrocardiography (ECG), Electromyography (EMG), Electroencephalogram (EEG), have relatively low-level amplitudes ranging from several μV (microvolts) to mV (millivolts). The frequency ranges of such biosignals are typically on the order of 0.1 Hz (Hertz) to 1KHz (kilohertz).

### SUMMARY

In an embodiment, there is provided an apparatus. The apparatus includes an analog front end for biosignal acquisition. The analog front end includes an instrumentation amplifier and a reconfigurable filter coupled to an output of the instrumentation amplifier. The instrumentation amplifier is configured to receive a biosignal. The instrumentation amplifier includes a super class-AB output stage. The reconfigurable filter has a selectable gain and an adjustable bandwidth. The bandwidth is adjusted based, at least in part, on a duty cycle of a clock signal.

In some embodiments, the apparatus further includes an analog to digital converter (ADC) and a duty cycle clock generator. The duty cycle clock generator is configured to provide the clock signal having the duty cycle to the reconfigurable filter and a second clock signal to the ADC.

In some embodiments of the apparatus, the biosignal is selected from the group including electrocardiogram (ECG), electromyogram (EMG) and electroencephalogram (EEG). In some embodiments of the apparatus, the analog front end further includes a programmable gain amplifier coupled between the instrumentation amplifier and the reconfigurable filter.

In some embodiments of the apparatus, the adjustable bandwidth is in the range of 100 Hertz (Hz) to 1 kilohertz (kHz). In some embodiments of the apparatus, the reconfigurable filter utilizes a switched-R-MOSFET-C (SRMC) topology.

In some embodiments of the apparatus, the instrumentation amplifier is a capacitively coupled chopper instrumentation amplifier. In some embodiments of the apparatus, the ADC includes a successive approximation register (SAR) configured to receive the second clock signal. In some embodiments of the apparatus, the ADC includes a switched capacitor digital to analog converter (DAC). In some embodiments of the apparatus, the reconfigurable filter and the ADC are fully differential.

In an embodiment, there is provided a front end integrated circuit (IC) for biosignal acquisition. The front end IC includes an analog front end, an analog to digital converter (ADC) and a duty cycle clock generator. The analog front end includes an instrumentation amplifier configured to receive a biosignal and a reconfigurable filter coupled to an output of the instrumentation amplifier. The instrumentation amplifier includes a super class-AB output stage. The reconfigurable filter has a selectable gain and an adjustable bandwidth. The bandwidth is adjusted based, at least in part, on a duty cycle of a clock signal. The duty cycle clock generator is configured to provide the clock signal having the duty cycle to the reconfigurable filter and a second clock signal to the ADC.

In some embodiments of the front end IC, the biosignal is selected from the group including electrocardiogram (ECG), electromyogram (EMG) and electroencephalogram (EEG). In some embodiments of the front end IC, the analog front end further includes a programmable gain amplifier coupled between the instrumentation amplifier and the reconfigurable filter. In some embodiments of the front end IC, the reconfigurable filter utilizes a switched-R-MOSFET-C (SRMC) topology. In some embodiments of the front end IC, the instrumentation amplifier is a capacitively coupled chopper instrumentation amplifier.

In some embodiments of the front end IC, the ADC includes a successive approximation register (SAR) configured to receive the second clock signal. In some embodiments of the front end IC, the ADC includes a switched capacitor digital to analog converter (DAC). In some embodiments of the front end IC, the reconfigurable filter and the ADC are fully differential.

In some embodiments of the front end IC, the adjustable bandwidth is in the range of 100 Hertz (Hz) to 1 kilohertz (kHz). In some embodiments of the front end IC, the adjustable bandwidth is in the range of 40 hertz (Hz) to 320 Hz with a 40 Hz step.

### BRIEF DESCRIPTION OF THE DRAWINGS

The drawings show embodiments of the disclosed subject matter for the purpose of illustrating features and advantages of the disclosed subject matter. However, it should be understood that the present application is not limited to the precise arrangements and instrumentalities shown in the drawings, wherein:

FIG. 1 illustrates a functional block diagram of a reconfigurable analog front end consistent with several embodiments of the present disclosure;

FIGS. 2A, 2B and 2C illustrate one example of the instrumentation amplifier of the reconfigurable analog front end of FIG. 1;

FIGS. 3A and 3B illustrate one example of the adjustable filter of the reconfigurable analog front end of FIG. 1;

FIG. 4 illustrates a sketch of a reconfigurable front end integrated circuit (IC) consistent with several embodiments of the present disclosure;

FIG. 5 illustrates a functional block diagram of one example implementation of the reconfigurable front end IC of FIG. 4;

FIG. 6A illustrates a schematic of an SAR (successive approximation register) control logic, e.g., SAR control logic of FIG. 5;

FIG. 6B illustrates a timing diagram of the SAR control logic, e.g., SAR control logic of FIG. 5;

FIG. 7A is a schematic of a current reuse two stage amplifier; and

FIG. 7B is a schematic of a dynamic common-mode feedback (CMFB) block configured to provide common mode feedback voltages V_{CMFB1} and V_{CMFB2} of FIG. 7A.

### DETAILED DESCRIPTION

Generally, this disclosure relates to a reconfigurable analog front end (AFE) for biosignal applications. The reconfigurable analog front end is configured to receive one or more biosignals. The biosignals may include, but are not limited to, electrocardiogram (ECG), electromyogram (EMG), electroencephalogram (EEG), arterial pressure wave (APW), etc. Reconfiguring the analog front end may facilitate utilizing a same analog front end in a variety of wearable health monitoring applications.

The reconfigurable analog front end includes an instrumentation amplifier and a filter. The reconfigurable analog front end may further include an analog-to-digital converter (ADC). The instrumentation amplifier includes a super class AB output stage configured to provide relatively high linearity. The filter has an adjustable gain and adjustable bandwidth configured to accommodate a variety of biosignals. The gain and bandwidth are adjusted utilizing a duty cycle control tuning. In an embodiment, the duty cycle controlled signal may be utilized by both the ADC and the filter. The reconfigurable analog front end, consistent with the present disclosure, is configured to have a relatively small form factor and to be relatively low power and thus appropriate for wearable applications.

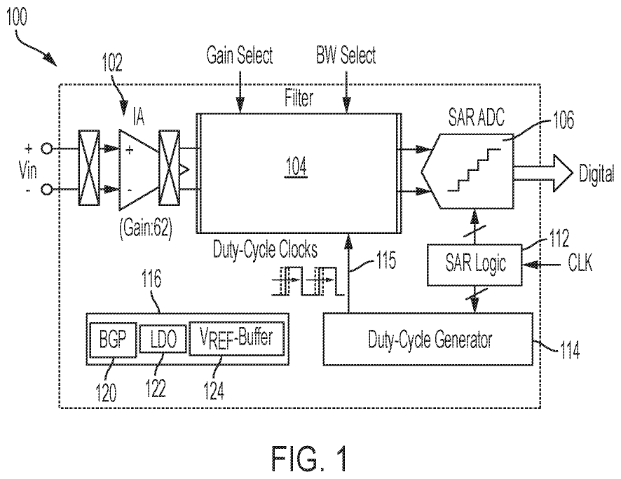

FIG. 1 illustrates a functional block diagram of a reconfigurable analog front end 100 consistent with several embodiments of the present disclosure. The reconfigurable analog front end 100 includes an instrumentation amplifier (IA) 102, an adjustable filter 104, and an ADC 106. The reconfigurable analog front end 100 may further include SAR logic 112, a duty cycle generator 114 and support circuitry 116. In an embodiment, the ADC 106 may correspond to a successive approximation register (SAR) ADC. In an embodiment, the support circuitry 116 may include one or more of a bandgap voltage reference (BGP) 120, a low dropout regulator (LDO) 122, and/or reference buffer 124.

Reconfigurable analog front end 100 is configured to receive an analog biosignal, Vin, as input. In one nonlimiting example, the biosignal input may correspond to a potential difference, i.e., a voltage. In another example, the biosignal input may be a differential input, i.e., Vᵢₙ=V+minus V−. The instrumentation amplifier 102 is configured to receive the analog biosignal, Vᵢₙ, as input and to provide a differential analog output to the filter 104. The filter 104 is configured to have an adjustable gain and an adjustable bandwidth controlled by a duty cycle of a duty cycle clock signal 115. In an embodiment, the SAR logic 112 may be re-used to drive the duty cycle generator 114 to generate one or more clock signals with a plurality of duty cycles. The different duty cycles may then be utilized to reconfigure the bandwidth and/or gain of adjustable filter 104.

A differential analog output of the filter 104 may then be provided to the ADC 106. The ADC 106 is configured to digitize the received output of filter 104 and the analog front end 100 may then provide a digital output corresponding to the differential analog input. In one nonlimiting example, SAR ADC 106 may correspond to a 10-bit fully differential SAR ADC configured to quantize a received differential input signal. The ADC 106 may be configured to sample at a rate of 2.5 KS (kilosamples)/s (second) to accommodate a 1 KHz maximum frequency input signal without aliasing. The ADC 106 is configured to implement a monotonic switching technique to reduce and/or minimize an area and power consumption of the reconfigurable analog front end 100. A synchronous SAR logic 112 is configured to avoid process, voltage, and temperature variations. To ensure high ADC linearity, a partial common-centroid layout strategy for a capacitor array may be utilized in the capacitor DAC (digital to analog converter). The reference voltage of the ADC 106 may be set to V_{DD}/2 by a dedicated on-chip reference buffer, giving an error voltage less than one LSB.

The reconfigurable analog front end 100 is thus configured to provide both a programmable gain (i.e., amplification) functionality and an adjustable filter functionality in one block. Such an architecture may then occupy a relatively smaller area and may have a relatively lesser power consumption. As used herein, the terms reconfigurable, programmable, adjustable and/or selectable are used interchangeably.

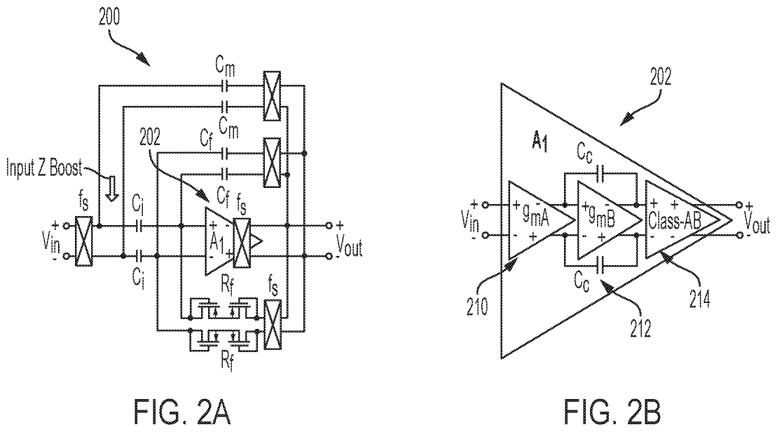

FIGS. 2A, 2B and 2C illustrate one example of the instrumentation amplifier 102 of the reconfigurable analog front end 100 of FIG. 1. In particular, FIG. 2A illustrates an architecture 200 of the instrumentation amplifier. FIG. 2B illustrates a block diagram of a core amplifier 202 of the instrumentation amplifier. FIG. 2C is a schematic 220 of one example circuitry of a two-stage amplifier and output buffer (e.g., super class AB output stage) corresponding to the core amplifier 202.

Turning first to FIG. 2A, the architecture 200 corresponds to a capacitively coupled chopper instrumentation amplifier (CCIA) topology that includes the core amplifier 202. The amplifier architecture 200 and CCIA topology are configured to optimize noise and power performance. The topology includes input capacitances, Cᵢ in series with the inverting and noninverting inputs of the core amplifier 202, feedback capacitors C_{f}, positive feedback capacitors Cₘ and feedback resistances R_{f}. A mid-band gain corresponds to a ratio of Cᵢ/C_{f} and a low cutoff frequency corresponds to 1/R_{f}C_{f}. The positive feedback capacitors C. are configured to increase a capacitive input impedance of the instrumentation amplifier.

Turning now to FIG. 2B, the core amplifier 202 includes a fully differential two-stage amplifier comprising a first stage 210 and a second stage 212. The first stage 210 is configured to receive the input biosignal Vᵢₙ and the second stage 212 is configured to provide a differential output. The core amplifier 202 further includes a buffer 214 corresponding to a class AB output stage. The buffer 214 is configured to provide a differential output Vₒᵤₜ.

In operation, the output of instrumentation amplifier 200 may be connected to a filter, a programmable gain amplifier or an analog to digital converter. It may be appreciated that each of these circuitries may provide a respective load to the instrumentation amplifier 200. Thus, the output stage is configured to handle variations in output load. In order to achieve a relatively high linearity, the core amplifier 202 may be configured to have a relatively high open loop gain.

Turning now to FIG. 2C, example amplifier 220 includes a first stage 222 and a second stage and buffer (super class AB output) combination 224. The first stage 222 corresponds to a gain stage. The second stage and buffer combination 224 are configured to operate in a super class AB operating mode, formed by a hybrid push pull topology and a complementary source follower topology. In one nonlimiting example, the two-stage amplifier may achieve a simulated DC open loop gain of 102 dB (decibels), a phase margin of 65° and a unit gain frequency of 50 kHz (kilohertz) resulting in a closed loop bandwidth of 1 kHz while dissipating 1.2 μA (microamperes) from a 1.8 V (volts) supply. The simulated total harmonic distortion of the instrumentation amplifier is higher than −80 dB with an input referred noise of 94 nV (nanovolts)/√Hz.

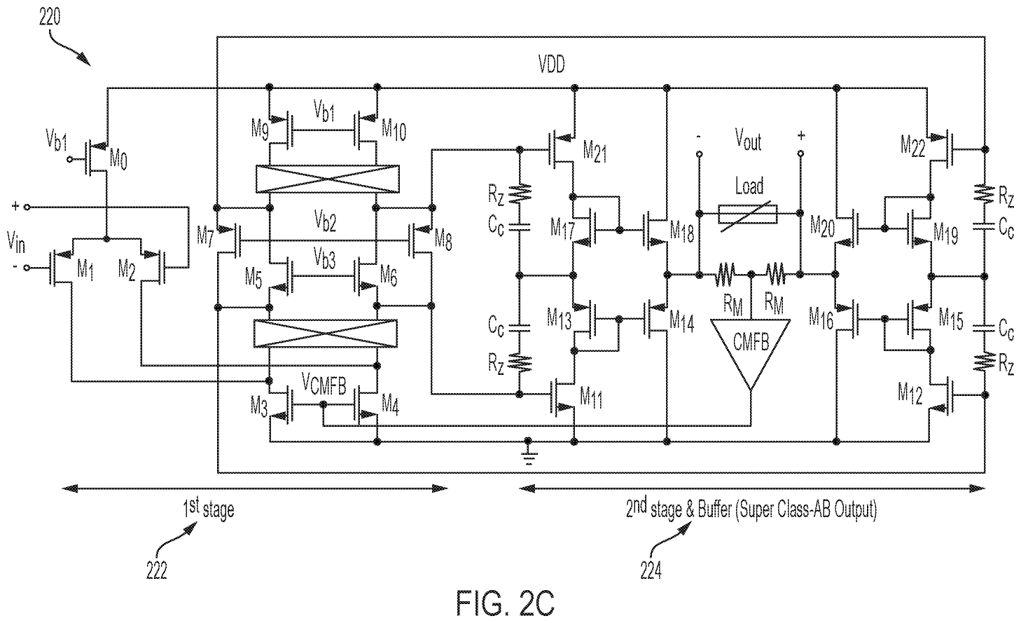

FIGS. 3A and 3B illustrate one example of the adjustable filter 104 of the reconfigurable analog front end 100 of FIG. 1. In particular, FIG. 3A illustrates an architecture 300 of the adjustable filter. FIG. 3B is a schematic 320 of one example circuitry of the core amplifier. The adjustable filter is configured to receive a differential input voltage, to amplify and low-pass filter the differential input voltage to yield an inverted differential output voltage. The amount of amplification (i.e., gain) is adjustable and a bandwidth (e.g., cutoff frequency) is also adjustable.

Turning now to FIG. 3A, the adjustable filter 300 includes a core amplifier 302, a pair 304 of adjustable input resistances, Rᵢ₂, a group 306 of switches (SW₁, SW₂, SW₃) a pair of feedback resistances, R_{f2}, and a pair of feedback capacitances, C. A first switch SW₁ is coupled between the inverting and noninverting inputs of the amplifier 302. A second switch SW₂ is coupled between the noninverting input and a first input resistance and a third switch SW₃ is coupled between the inverting input and a second input resistance.

The filter 300 utilizes a switched-R-MOSFET-C (SRMC) topology. Rᵢ₂, R_{f2}, C and the core amplifier 302 are configured to form a feedback loop. Switches SW₁, SW₂, SW₃ are driven by a set of complementary clocks Φ and Φb with respective clock duty cycles. A transfer function of the filter 300 may be written as:

$$H ( s ) = \frac{{V}_{out}}{{V}_{in}} \approx \frac{- \frac{{R}_{f 2}}{{R}_{i 2}}}{1 + \frac{s}{{ω}_{cutoff}}} \qquad ( 1 )$$

where the −3 dB cutoff frequency can be written as

$${ω}_{cutoff} = \frac{d}{{R}_{f 2} C} ,$$

and d is the duty-cycle ratio. As indicated by Eq. (1), the bandwidth of the filter 300 may be tuned by adjusting the duty-cycle ratio, d. Filter 300 gain (R_{f2}/Rᵢ₂) may be adjusted by adjusting Rᵢ₂. For relatively accurate bandwidth control, the duty-cycle generator 308 may be configured to utilize the SAR logic (e.g., SAR logic 112 of FIG. 1) to generate a series of clock duty cycles with d adjusted from 0.083˜0.83. This range of duty cycles is configured to achieve a programmable bandwidth of 100˜1 KHz with a step of 100 Hz, and gain settings of 2/3/4/5.

A state of the first switch SW₁ is controlled by a first control input signal, Φb, and states of the second switch SW₂ and the third switch SW₃ are controlled by a second control input signal, Φ. The control input signals are provided by signal source 308. For example, signal source 308 may correspond to duty cycle generator 114 of FIG. 1. In other words, an amount of time that a switch is open or closed is related to a duty cycle ratio of each of the first and second control input signals. A duty cycle (d) of the second control input signal is related to a cutoff frequency and thus the bandwidth of the adjustable filter 300. In one nonlimiting example, the duty cycle (d) may be in the range of 0.083 to 0.83.

In an embodiment, adjustable filter 300 is configured to have a −3 dB cutoff frequency in the range of 100 Hz to 1 kHz. This range of cutoff frequencies corresponds to an adjustable passband configured to pass a plurality of biosignals. In other words, a selection of a specific cutoff frequency and thus passband bandwidth may be based, at least in part, on the particular biosignal (i.e., ECG, EMG, EEG). Selecting the cutoff frequency by selecting a duty cycle ratio of the control input signal allows a same reconfigurable analog front end to be used in a plurality of biosignal applications.

Turning now to FIG. 3B, core amplifier 320 is one example of core amplifier 302 of FIG. 3A. Core amplifier 320 includes a folded-cascode input stage combined with a class-AB output stage. By sensing the output voltage at Vₒᵤₜ⁺ and Vₒᵤₜ⁻, a continuous common-mode feedback (CMFB) sets the common mode voltage V_{CMFB}. The sizes of the input transistors may be increased to reduce the noise of the amplifier 320. The amplifier 320 may achieve a simulated DC open-loop gain of 75 dB, phase margin of 70 degrees and a unit-gain frequency of 14 KHz, resulting in a closed-loop bandwidth of 1 KHz while dissipating 0.4 μA from a 1.8V supply. Because of the class-AB output stage, the amplifier 320 has a rail-to-rail output swing.

Thus, using a low-noise IA with a high-driving super class-AB output stage, an AFE consistent with the present disclosure may achieve a −1.6 dB THD (total harmonic distortion) with 2.88 μW of dc power consumption in the AFE. Utilizing tunable duty-cycle clocks to drive a reconfigurable filter, the AFE is configured to achieve programmable gains of 42-50 dB and an adjustable bandwidth of 100 Hz-1 KHz with 100 Hz steps. Thus, this method can achieve as low as 100 Hz bandwidth without the penalty of large resistors/capacitors and large chip area. The reconfigurability features also enable both tuning antialiasing filtering and wide dynamic range, allowing the following ADC to produce a high-fidelity signal. The corresponding integrated circuit (IC, i.e., “chip”) occupies an active area of 0.625 mm². An AFE consistent with the present disclosure may be configured to achieve relatively high linearity with a competitive performance in power consumption, noise, and chip area.

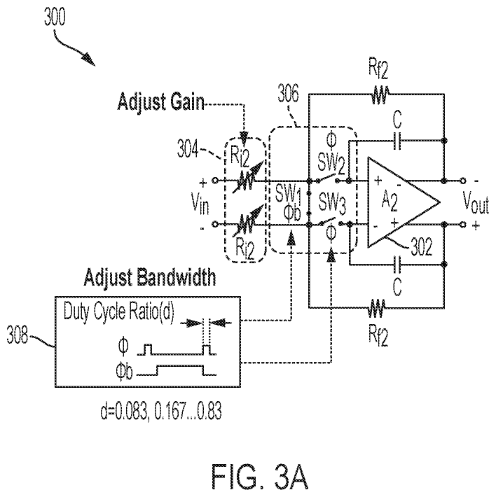

FIG. 4 illustrates a sketch of a reconfigurable front end integrated circuit (IC) 400 consistent with several embodiments of the present disclosure. The reconfigurable front end IC 400 may be utilized for acquiring one or more low frequency biomedical signals, e.g., biosignals. The reconfigurable front end IC 400 includes an instrumentation amplifier (IA) 402, a programmable gain amplifier (PGA) 404, an adjustable bandwidth filter 406 and an ADC 408. Similar to the analog front end 100 of FIG. 1, bandwidth of the filter 406 may be adjusted based, at least in part, on one or more clock signals 410 (e.g., duty cycles). The clock signals 410 may be used by both the ADC 408 and the filter 406. In one nonlimiting example, the filter 406 may correspond to an SRMC filter, as described herein. The bandwidth of the filter 406 may be adjusted based, at least in part, on a duty cycle of the clock signal. The ADC may be configured to receive a second clock signal. Both clock signals may be provided by a single SAR logic/clock cycle generator.

The reconfigurable front end IC 400 implements a filter-ADC architecture employing an SRMC programmable filter 406 in a biomedical FE circuit 400, with the SAR control logic in the ADC reused as the duty-cycle control clock configured to provide, e.g., clock signals 410. The SAR ADC 408 may be configured to provide a clock signal corresponding to a series of clock pulses to the SRMC filter (i.e., low pass filter (LPF)). The clock pulses 410 are configured to have a duty-cycle ratio ranging from 0.1-0.8. Utilizing the clock signals from the ADC 408 allows the filter 406 to achieve a smallest bandwidth of 40 Hz without the penalty of large passive components and obviates the need for an additional clock generator. The SRMC filter 406 may then utilize one (Rᵢ−R_{f}−C_{f}) design-set for bandwidth tunability, thus reducing chip area. In one nonlimiting example, an AFE utilizing the filter-ADC architecture in a complete biosignal acquisition system may achieve a −68 dB THD and a 66 dB DR through the use of bandwidth tuning within a feedback loop.

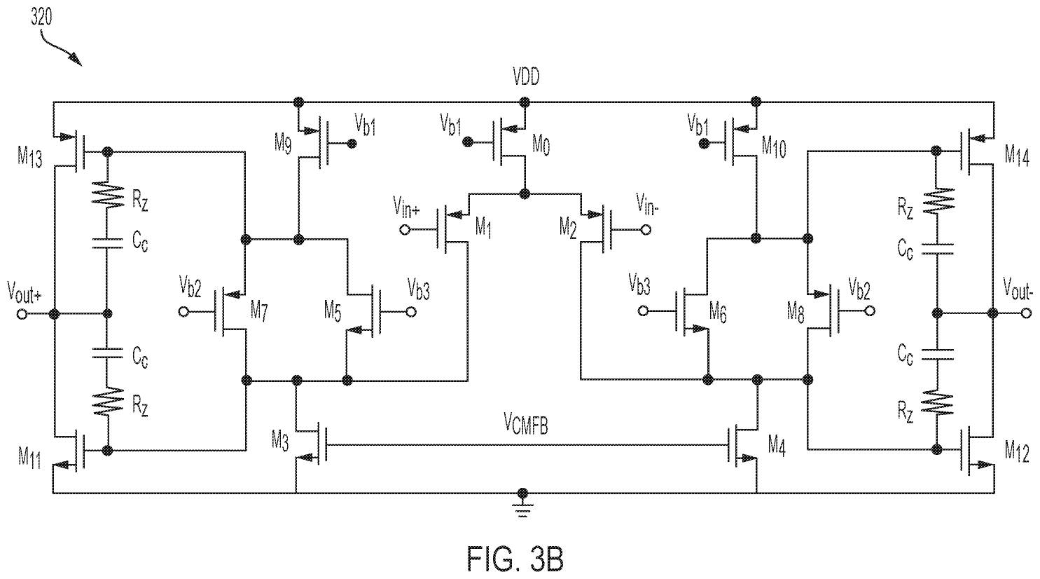

FIG. 5 illustrates a functional block diagram 500 of one example implementation of the reconfigurable front end IC 400 of FIG. 4. Reconfigurable front end IC 500 includes an analog front end 501 and an SAR ADC 508. The AFE 501 includes an instrumentation amplifier (IA) 502, a programmable gain amplifier (PGA) 504 and an adjustable SRMC filter 506. The IA 502 includes an IA core amplifier 503, the PGA 504 includes a PGA core amplifier 505 and the SRMC filter 506 includes a filter core amplifier 507.

The SRMC circuit 506 includes a feedback capacitor C_{f} across the filter core amplifier 507, a resistive feedback network Rᵢ and R_{f} and a plurality of switches S₁, S₁, S₂. Each switch S₁ is configured to selectively couple an input resistance Rᵢ to a corresponding input to the core amplifier 507. S₂ is configured to selectively couple the inverting and noninverting inputs to the core amplifier 507. Switches S₁ and S₂ are driven by non-overlapping clock signals (“clocks”) Φ and Φ̅, respectively. The clock signals are provided from SAR control logic 514 that is included in SAR ADC 508. The period of Φ is T=2π/ω_{S}, where ω_{S} corresponds to a switching frequency. The ratio of the turn-on time (tₒₙ) to T is defined as the duty-cycle ratio (d). The ON-resistance of S₁ is assumed to be small and may be neglected. The SRMC circuit 506 may operate as either a sample-and-hold (S/H) circuit or a filter, depending on a relative relationship between a cutoff frequency ω_{RC} and the switching frequency ω_{S}. In particular, SRMC circuit 506 may operate as an S/H circuit when ω_{S}<ω_{RC} and as a filter with a tunable bandwidth when ω_{S}>2ω_{RC}. When the SRMC circuit 506 is operating as an S/H circuit, the transfer function of the SRMC circuit corresponds to a low pass filter with passband gain R_{f}/Rᵢ and a bandwidth ω_{RC}=1/(R_{f}C_{f}).

When the SRMC circuit 506 is operating as a filter with tunable bandwidth, the bandwidth corresponds to:

$${ω}_{3 dB} = \frac{d}{{R}_{f} {C}_{f}} \qquad ( 2 )$$

where d is the duty-cycle ratio (0<d≤1) of Φ. It may be appreciated that a duty-cycle controlled filter configuration, consistent with the present disclosure, may facilitate a reduced size of passive components included in the filter 506. For example, to achieve a bandwidth of 40 Hz, a conventional first-order active-RC filter may utilize a C_{f} of 80 pF and an R_{f} of 50 MΩ. An SRMC circuit with a d=0.1 may utilize C_{f}=80 pF and R_{f}=5 MΩ, a 90% reduction in the resistor size compared to the conventional active-RC filter. SRMC circuit 506 is configured to achieve tunable bandwidth with one resistor and one capacitor combination.

The filtering action may be understood as an insufficient transient in the sampling phase (Φ=1), i.e., the sampling frequency ω_{S} is so high that the capacitor voltage may be unable to follow the input signal within the short sampling phase. For Φ=1, S₁ is on, and the current Vᵢₙ/Rᵢ is shared between R_{f} and C_{f}, When Φ=0, S₂ is on, and Vᵢₙ/Rᵢ is directed to ground. Thus Vᵢₙ, Rᵢ, S₁ and S₂ can be modeled as an equivalent current source (d·Vᵢₙ/Rᵢ)

In terms of noise performance, the total output noise power may be a constant value that depends on C_{f}, and does not vary with d. The tunable-bandwidth function is configured to filter out the noise from previous stages (e.g., IA 502 and PGA 504), but does not affect the filter's noise. The previous stages generally have a relatively large gain, thus the filter's noise is relatively small and may be ignored.

Turning now to the combination of filter 506 and ADC 508, a parasitic capacitance may exist between the inverting input of the filter core amplifier 507 and ground. ADC 508 includes a comparator 509, SAR control logic 514 and sampling capacitor arrays, i.e., switched capacitor digital to analog converters (DACs), 516 and 518. A capacitance of each switched capacitor array 516, 518 corresponds to C_{DAC} and is controlled by clock signals CLK1˜CLK8 output from SAR control logic 514. A differential output of filter core amplifier 507 (and thus filter 506) is selectively coupled to a differential input of the ADC 508 via a pair of sampling, i.e., sample and hold (S/H), switches, both indicated as S_{S/H}. A positive side of the filter differential output voltage, Vₒᵤₜ, is selectively coupled to a first (positive, in this configuration) capacitive DAC 516 and a negative side of the filter differential output voltage, Vₒᵤₜ, is selectively coupled to a second (negative, in this configuration) DAC 518. A first output (V_{DACP}) of the first DAC 516 is coupled to the noninverting input of comparator 509 and a second output (V_{DACN}) of the second DAC 518 is coupled the inverting input of comparator 509.

SAR control logic 514 is configured to provide a sampling clock signal, Φ_{S}, to the sampling switches S_{S/H}. Generally, the clock signals (i.e., waveforms) Φ and Φ̅ may both be off or one or the other may be on. The clock waveform Φ_{S} may correspond to clock waveform Φ̅ and is configured to allow the filter output to be sampled within an acquisition time t_{acq}, where t_{acq} corresponds to the on time of the sample clock signal Φ_{S}.

When considering the time response, in one conversion cycle, the operation of the SRMC filter 506 together with the SAR ADC (i.e., S/H circuit) 508 can be divided into two phases. For a first phase and time interval 0<t<t₁, S1 is ON, S2 and S_{S/H} are OFF. In this first phase, Vₒᵤₜ of the filter 506 is configured to track Vin, and C_{DAC} may hold the filter's output voltage from a previous phase. At t=t₁, S1 turns off (Φ goes low), and the output voltage just before time t₁ may be represented as Vₒᵤₜ(t₁₋).

For a second phase and time interval t₁<t<T, S1 is OFF, S2 and S_{S/H} are ON. In this phase, the SRMC filter 506 may hold the voltage on C_{f}, and the ADC 508 is configured to sample the signal onto C_{DAC}. It may be appreciated that, in the second phase, the ADC 508 and its sampling noise may be isolated from input voltage Vᵢₙ. At t=t₁, S_{S/H} closes, S1 opens, and the charge is shared between C_{f} and C_{DAC}. According to charge conservation, Vₒᵤₜ may drop by ΔV, where:

$$Δ V \approx \frac{{C}_{DAC}}{{C}_{f} + {C}_{DAC}} \cdot {V}_{out} ( {t}_{1 -} ) \qquad ( 3 )$$

Since there is no charging/discharging path at the inverting input of the filter amplifier 507, V− also drops by ΔV at t=t₁, and yields a positive voltage difference ΔV at the amplifier input. Thus, Vₒᵤₜ follows a linear settling behavior of Vₒᵤₜ(t)=Vₒᵤₜ(t₁₋)−ΔV·exp^{-(t-t1)/τ}, where τ=1 is the time constant of the filter core amplifier 507 with a load capacitance of (C_{DAC}+Cf∥Cp).

To settle within an error of ε_{dynamic}, the acquisition time t_{acq} should satisfy:

$${t}_{acq} \geq τ \cdot {ln}^{\frac{Δ V}{{ε}_{dynamic}}} \qquad ( 4 )$$

Therefore, a larger ΔV would require a higher fₜ to ensure that the voltage ε_{dynamic} is small enough within t_{acq}.

When considering the frequency response, the SRMC filter 506 behaves as a first-order LPF (low pass filter) with a cutoff frequency of d/R_{f}C_{f} (from Eq. (2)), and has a closed-loop transfer function of

$${A}_{closed} \approx ( - \frac{{R}_{f}}{{R}_{i}} ) \cdot [ 1 - \frac{1}{{A}_{0}} \cdot ( 1 + \frac{{R}_{f}}{{R}_{i}} ) ] \qquad ( 5 )$$

where A₀ is the open-loop gain of the core amplifier 507. The filter 506 achieves an inverting gain of −R_{f}/Rᵢ with a static error of

$${ε}_{static} = [ \frac{1}{{A}_{0}} \cdot ( 1 + \frac{{R}_{f}}{{R}_{i}} ) ] .$$

To connect to an N-bit ADC with a reference voltage V_{REF}, the total error ε=ε_{dynamic}+ε_{static} should be lower than ½ LSB, where LSB=V_{REF}/2^{N} is the least-significant-bit of the ADC. For the combination of SRMC filter 506 and SAR ADC 508, both the dynamic error and static error are generally within ¼ LSB.

An SRMC filter together with ADC may be designed, as described herein. For example, for the feedback capacitance C_{f} to satisfy the Nyquist limit and Eq. (2), C_{f} may be determined as:

$${C}_{f} > \frac{d}{π \cdot {R}_{f} \cdot {f}_{s}} \qquad ( 6 )$$

In one nonlimiting example, for f_{S}=1−kS/s, R_{f}=5 MΩ and d set to its maximum value of 1, using Eq. (6), C_{f} should be larger than 64 pF. For example, C_{f} may be selected as 80 pF to achieve a 400 Hz cutoff frequency.

Similarly, from Eq. (5), and with Rᵢ=2 MΩ and R_{f}=5 MΩ as examples, to get an error term below 0.1%, the open-loop gain A₀ should be higher than 71 dB. For the unity-gain frequency fₜ of the SRMC filter 506, ΔV is a function of the input signal from Eq. (3), thus ε_{dynamic} also depends on the signal level. Considering the worst case scenario, when Vₒᵤₜ(t₁₋) is at its largest allowable value of V_{REF}/2, Vₒᵤₜ should settle within ¼ LSB of its final value in the acquisition time t_{acq}. The minimum fₜ can be expressed as:

$${f}_{t} \geq \frac{1}{{t}_{acq}} {ln}^{[ ( \frac{{C}_{DAC}}{{C}_{DAC} + {C}_{f}} ) \cdot {2}^{N + 1} ]} \qquad ( 7 )$$

From Eq. (7), the relationship between the effective number of bits (ENOB) and fₜ in different t_{acq} may be simulated. It may be appreciated that a smaller t_{acq} corresponds to a higher fₜ to maintain the target ENOB. For example, if C_{DAC}=10 pF and C_{f}=80 pF, an 8-bit SAR ADC with a t_{acq} of 0.1 ms (milliseconds) corresponds to a minimum fₜ of 40 kHz.

Thus, a fully-differential architecture of the SRMC filter 506 and the SAR ADC 508 may be used for high common mode rejection ratio (CMRR), power supply rejection ratio (PSRR), and a wide signal swing. The SAR control logic circuit 514 may be re-utilized as a duty-cycle clock generator for the SRMC filter 506, as described herein. Table 1 includes a summary of parameters, equations and target values for a reconfigurable front end IC, as described herein.

TABLE 1

| | | |
|---|---|---|
| Specification | Equation | Target |

ADC

| | | |
|---|---|---|
| Resolution | N-bit | 8-bit |
| Sampling Rate | f_{S} | 1-kS/s |
| Acquisition Time | t_{acq} | 0.1 ms |
| Sampling Cap. | C_{DAC} | 10 pF |
| Ref. Voltage | V_{REF} | 1 V |

SRMC Filter

| | | |
|---|---|---|
| Condition | ${f}_{S} > 2 \cdot \frac{1}{2 π {R}_{f} {C}_{f}}$ | 1-kS/s > 2 · 400 Hz |
| Gain (A_{closed}) | ${A}_{closed} = - \frac{{R}_{f}}{{R}_{i}}$ | $- \frac{5 M Ω}{2 M Ω} = - 2.5$ |
| BW (f_{3 dB}) | $\frac{d}{2 π {R}_{f} {C}_{f}} , 0 < d \leq 1$ | 40-400 Hz |
| Output Swing | V_{REF} | 1 V |
| Cap. (C_{f}) | ${C}_{f} > \frac{d}{π \cdot {R}_{f} \cdot {f}_{s}}$ | >64 pF @ d = 1 |
| Output Noise | $( \frac{K T}{{C}_{f}} ) \cdot ( 1 + \frac{{R}_{f}}{{R}_{i}} )$ | 181 pVᵣₘₛ² |

The Core Amplifier of the SRMC Filter

| | | |
|---|---|---|
| Gain (A₀) | ${A}_{0} > {2}^{N + 2} \cdot ( 1 + \frac{{R}_{f}}{{R}_{i}} )$ | >71 dB |
| fₜ (unity gain freq.) | $\geq \frac{1}{{t}_{acq}} {ln}^{[ ( \frac{{C}_{DAC}}{{C}_{DAC} + {C}_{f}} ) \cdot {2}^{N + 1} ]}$ | ≥40 kHz |

In an embodiment, the SAR ADC 508 may be configured to have an 8-bit resolution and a 1-kS/s sampling rate. A monotonic switching structure may be employed configured to reduce the power consumption and the size of the sampling capacitor C_{DAC} (of capacitive DACs 516, 518). In one nonlimiting example, to achieve a relatively high linearity of the ADC, the sampling capacitor C_{DAC}, including capacitors C₀₋₇, may be selected as 10 pF. Once the output signals from the SRMC filter 506 are sampled onto C₀₋₇, the comparator 509 is configured to compare the voltages at V_{DACP} and V_{DACN}. An output of the comparator 509 is input to the SAR control logic 514. The SAR control logic 514 may then be configured to adjust the two DACs 516, 518. For an 8-bit resolution, this sample-compare-adjust process is configured to repeat for 8 cycles until the output voltage difference between the two DACs 516, 518 is less than 1 LSB. To implement the conversion process, the SAR control logic 514 is configured to adjust the control clocks Φ_{S} and CLK₁₋₈. Φ_{S} is the clock to control the switch S_{S/H} and CLK₁₋₈ are the signals to control the DACs and the SRMC filter 506, as described herein.

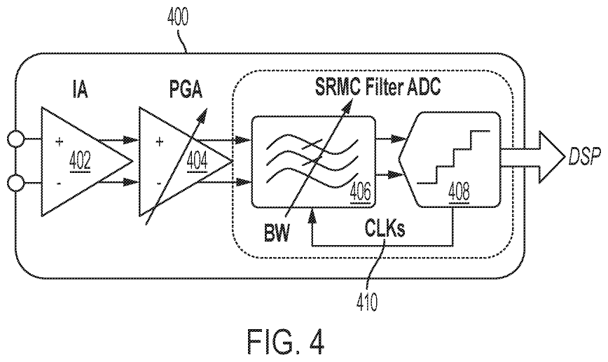

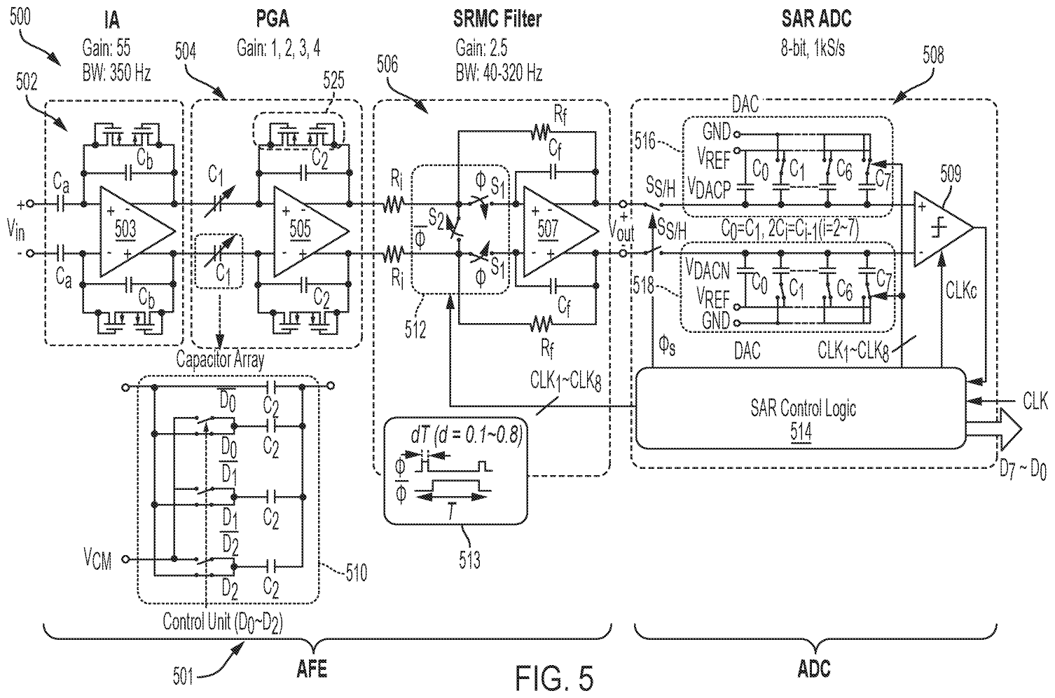

FIG. 6A illustrates a schematic 600 of the SAR control logic, e.g., SAR control logic 514, of FIG. 5. FIG. 6B illustrates a timing diagram 650 of the SAR control logic, e.g., SAR control logic 514, of FIG. 5. FIGS. 6A and 6B may be best understood when considered in combination with FIG. 5. In an embodiment, the SAR control logic 514 may be implemented as a synchronous architecture configured to generate relatively accurate clock signals. A synchronous ADC architecture may be relatively more robust to PVT variations compared to an asynchronous architecture. The clock generation, anti-aliasing filtering and digitization function may be integrated into one filter-ADC block (e.g., filter 506 and ADC 508), thus eliminating power/area consumption from a dedicated clock generation block.

The control logic 600 includes a number, e.g., 9, D-flip flops (DFFs) 602 combined to form a shift register. A falling edge pulse on a reset signal input (RST) is configured to trigger the SAR control logic 600. A time duration of one conversion corresponds to ten cycles of system clock CLK. After Φ_{S} resets all DFFs, the system clock CLK is configured to trigger a series of DFFs to generates CLK₁₋₈ successively. In one nonlimiting example, for a CLK with frequency 10-kHz, the acquisition time t_{acq} is 0.1 ms.

The SAR control logic timing diagram 650 includes the system clock signal CLK, reset input signal RST, the switching control signal Φ_{S} and each clock control signal CLK₁-CLK₈. Clock signals CLK₁-CLK₈ may be re-utilized as the clock source of Φ in the SRMC filter 506. CLK₁ to CLK₈ are configured to provide a duty-cycle ratio range of 0.1-0.8, thus the SRMC filter 506 may achieve 8 programmable bandwidths f_{3 dB} from 40 Hz to 320 Hz with a 40 Hz frequency step. Generalizing this idea, an N-bit SAR ADC may achieve N bandwidths in the corresponding filter, where d is given by:

$$d = \frac{i}{N + 2} , ( i = 1 , 2 , … , N ) \qquad ( 8 )$$

It may be appreciated that, according to Eq. (8), as the resolution of the ADC increases, a number of steps for f_{3 dB} also increases (from Eq. (2)). Thus, if the filter bandwidth is set relatively closer to the signal bandwidth, there may be relatively less noise from the overhead frequency range. Re-utilizing the clocks CLK₁₋₈, as described herein, is configured to eliminate an additional duty-cycle clock generator and simplify connections to the filter and the SAR ADC (e.g., S/H circuit).

In one nonlimiting example, with Rᵢ=2 MΩ (Mega-ohms), R_{f}=5 MΩ, and C_{f}=80 pF (pico-farads), SRMC filter 506 may achieve a gain of 2.5 and an RC bandwidth f_{RC}=400 Hz. To avoid having a relatively large turn-on resistance, switches S1 and S2 in the filter 506 may correspond to a transmission-gate switch (CMOS switch). Continuing with this example, the turn-on resistance is 10 kΩ. In another example, for a 1 V reference voltage (V_{REF}) in the ADC 508, the SRMC filter 508 may have an output signal swing of 2 V_{PP} (volts peak to peak), corresponding to a wide input signal swing of 0.8 V_{PP}.

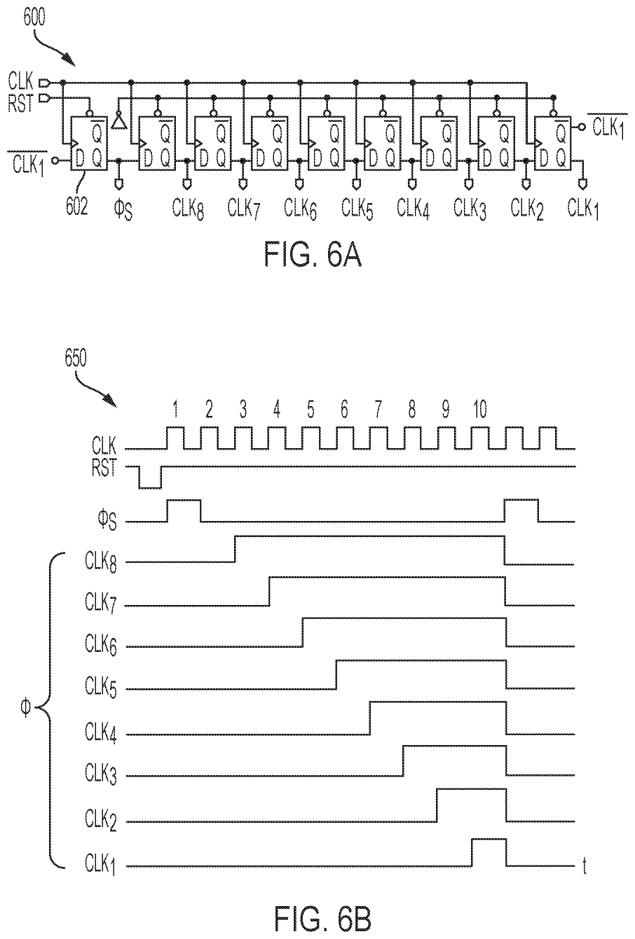

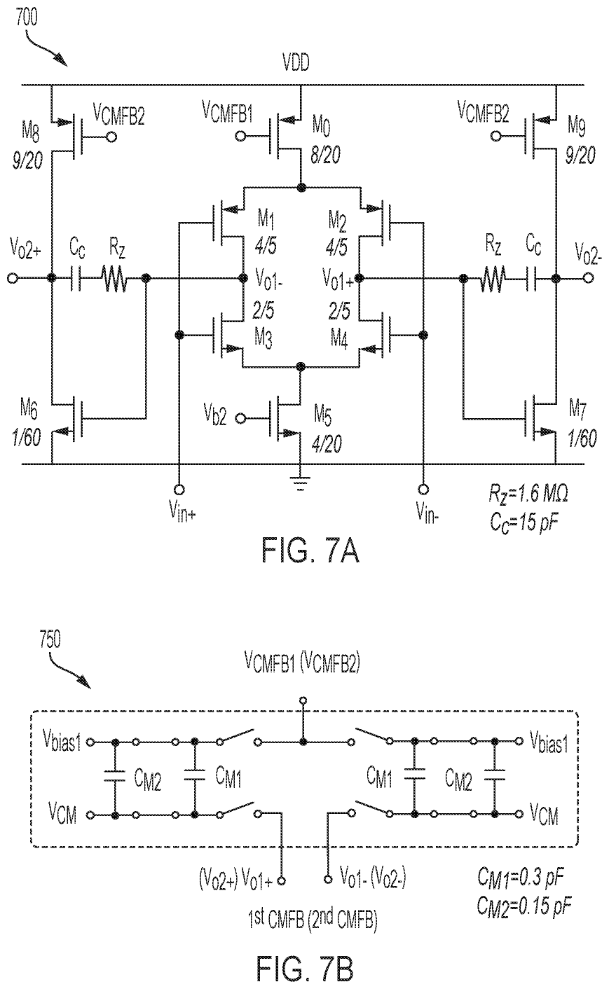

FIG. 7A is a schematic 700 of a current reuse two stage amplifier. Amplifier 700 is one example of filter core amplifier 507 of FIG. 5. A resolution of a digitization process may depend on a unity-gain frequency of a filter core amplifier. For example, a relatively high unity-gain frequency may correspond to relatively better resolution of the digitization process. The current-reuse two-stage amplifier 700 includes a first stage formed of M₀₋₅ that has a complementary input stage. The current-reuse two-stage amplifier 700 includes a second stage formed of M₆₋₉ that realizes an output buffer. The complementary input stage provides a doubled transconductance to achieve a relatively high unity-gain frequency

$${f}_{t} = \frac{{gm}_{1} + {gm}_{3}}{2 π \cdot {C}_{C}}$$

and a relatively low input-referred noise of

$$\frac{\overline{{V}_{nl}^{2}}}{Δ f} = \frac{16}{3} \frac{1}{{gm}_{1} + {gm}_{3}} .$$

FIG. 7B is a schematic 750 of a dynamic common-mode feedback (CMFB) block configured to provide common mode feedback voltages V_{CMFB1} and V_{CMFB2} of FIG. 7A. Two dynamic common-mode feedback (CMFB) blocks, e.g., two of dynamic common-mode feedback block 750, may be implemented in each stage of current reuse two stage amplifier 700. The dynamic CMFB blocks 750 are configured to provide a stable DC biasing. The CMFB 750 is configured to sense at Vₒ₁₊ (Vₒ₂₊) and Vₒ₁₋ (Vₒ₂₋), and feedback to V_{CMFB1} (V_{CMFB2}).

In one nonlimiting example, simulation results show that the amplifier 700 achieves an open-loop gain (A₀) of 74 dB and an unit-gain frequency (fₜ) of 58 kHz with a phase margin of 65°, which meets the target values included in Table I. Amplifier 700 achieves an input-referred noise of 72 nV/√Hz with a 0.9 μA current consumption from a 2-V dc supply. The amplifier 700 has an A₀ larger than 69 dB at 2 V_{PP} differential output. The amplifier 700 can maintain a stable A₀ of 74 dB at a 1.5 V_{PP} differential output. Utilizing Eq. (5), the corresponding static error (ε_{static}) for the SRMC filter is larger than 0.06%, resulting in a relatively high linearity performance over a relatively wide signal DR (dynamic range).

Turning again to FIG. 5, example front end IC 500 includes low-noise IA 502, PGA 504, SRMC filter 506 and SAR ADC 508, as described herein. Both the IA 502 and PGA 504 use a capacitor-feedback topology configured to achieve a low-noise performance. In one nonlimiting example, the IA 502 may have a V_{IRN} of 3.2 μVᵣₘₛ over a bandwidth of 0.9-350 Hz with a midband gain of 55, and a THD of 0.06% at a 5.5 mV_{PP} input signal. “IRN” corresponds to input-referred noise. The IA 502 may include a 154 dB open-loop gain two-stage class-AB amplifier configured to achieve a high-linearity performance.

The PGA 504 includes capacitive feedback formed by C₁, i.e., capacitor array 510, and C₂, and a pseudoresistor (e.g., formed of transistors 525). The PGA 504 may be configured to utilize the same core amplifier as the SRMC filter, e.g., filter core amplifier 506. The capacitive feedback is configured to prevent the amplifier's offset voltage from saturating the output stage. In one nonlimiting example, by adjusting the capacitor array C₁, the PGA 504 may implement gain settings of 1, 2, 3, and 4. It may be appreciated that when the signal path is disabled, the unit-capacitor C₂ in the capacitor array C₁ 510 is configured to switch an input side of the capacitor to a virtual ground (V_{CM}) instead of floating it, thus avoiding signal leakage from the IA 502 when the switch is off.

Analog front end IC 500 that includes SRMC filter 506 is configured to provide a programmable-bandwidth function to suppress out-of-band noise. The V_{IRN} may be determined as:

$${V}_{IRN , FE} \approx \sqrt{\frac{\overline{{V}_{nl , FE}^{2}}}{Δ f} \cdot \frac{π}{2} \cdot {f}_{3 dB}} \approx \sqrt{\frac{\overline{{V}_{nl , IA}^{2}}}{Δ f} \cdot \frac{d}{4 {R}_{f} {C}_{f}}} \qquad ( 9 )$$

where f_{3 dB}=ω_{3 dB}/2π can be obtained from Eq. (2), and

$$\frac{\overline{{V}_{nl , FE}^{2}}}{Δ f} \approx \frac{\overline{{V}_{nl , IA}^{2}}}{Δ f}$$

due to the high-gain of IA. Thus, a reduced duty-cycle ratio d would effectively reduce the in-band noise of the analog front end IC.

Thus, in an embodiment consistent with the present disclosure, an analog front end IC, e.g., analog front end IC 500, may be implemented in 0.13 μm CMOS technology. The AFE 501 may include an IA, a PGA, and a SRMC filter, as described herein. In this example, thick-oxide 3.3 V FET devices are used throughout the AFE 501 since they are more ESD (electrostatic discharge) robust. Robustness against ESD is important in medical applications where the circuits suffer from the electrostatic charge from the human body. Thick-oxide FETs may have relatively less gate leakage current, which is more suitable for pseudo-resistor implementation in the IA and PGA. The complete system (i.e., analog front end IC 500) occupies 0.6 mm² active area, while consuming 6.3 μW dc power from a 2-V supply. To achieve the design targets in Table I, the SRMC filter and ADC consume 27% and 19% of the system power, respectively.

Thus, a reconfigurable front-end (FE) circuit for acquiring various low-frequency biomedical signals has been described. An energy and area-efficient tunable filter is configured to adapt a FE bandwidth to the signal of interest. The filter may utilize a switched-R-MOSFET-C (SRMC) technique to realize an ultra-low cutoff frequency. An 8-bit SAR ADC, following the filter, is configured to quantize the signal. The SAR control logic is re-used to accurately program the filter bandwidth from 40 Hz to 320 Hz with a 40 Hz step. A prototype integrated circuit includes the FE circuit, formed of an instrumentation amplifier (IA), a programmable-gain amplifier (PGA), and the tunable filter followed by the SAR ADC. Implemented in 0.13 μm CMOS technology, the IC occupies a 0.6 mm² active area while consuming 6.3 μW dc power from a 2-V supply. Measurement results show a FE gain range of 43-55 dB with an integrated input-referred noise (V_{IRN}) of 3.45 μVᵣₘₛ, a 66 dB dynamic range (DR), and a total-harmonic distortion (THD) of −68 dB at an input amplitude of 6 mV_{PP}. The effective number of bits (ENOB) of the ADC is 7.921 bits at 1-kS/s. In real-time Electrocardiogram (ECG), Electromyography (EMG), and Electroencephalography (EEG) measurements, high-fidelity waveforms are acquired using the proposed FE IC, validating the system's reconfigurability and relatively high-linearity.

“Circuitry”, as used in any embodiment herein, may include, for example, singly or in any combination, hardwired circuitry, programmable circuitry such as computer processors including one or more individual instruction processing cores, state machine circuitry, and/or firmware that stores instructions executed by programmable circuitry. The logic may, collectively or individually, be embodied as circuitry that forms part of a larger system, for example, an integrated circuit (IC), an application-specific integrated circuit (ASIC), a field-programmable gate array (FPGA), a programmable logic device (PLD), a complex programmable logic device (CPLD), a system on-chip (SoC), etc.

1. An apparatus comprising:
an analog front end for biosignal acquisition, the analog front end comprising:
an instrumentation amplifier configured to receive a biosignal, the instrumentation amplifier comprising a super class-AB output stage; and
a reconfigurable filter coupled to an output of the instrumentation amplifier, the reconfigurable filter having a selectable gain and an adjustable bandwidth, the bandwidth adjusted based, at least in part, on a duty cycle of a clock signal.

2. The apparatus of claim 1, further comprising an analog to digital converter (ADC) and a duty cycle clock generator, the duty cycle clock generator configured to provide the clock signal having the duty cycle to the reconfigurable filter and a second clock signal to the ADC.

3. The apparatus of claim 1, wherein the biosignal is selected from the group comprising electrocardiogram (ECG), electromyogram (EMG) and electroencephalogram (EEG).

4. The apparatus of claim 1, wherein the analog front end further comprises a programmable gain amplifier coupled between the instrumentation amplifier and the reconfigurable filter.

5. The apparatus of claim 1, wherein the adjustable bandwidth is in the range of 100 Hertz (Hz) to 1 kilohertz (kHz).

6. The apparatus of claim 1, wherein the reconfigurable filter utilizes a switched-R-MOSFET-C (SRMC) topology.

7. The apparatus of claim 1, wherein the instrumentation amplifier is a capacitively coupled chopper instrumentation amplifier.

8. The apparatus of claim 2, wherein the ADC comprises a successive approximation register (SAR) configured to receive the second clock signal.

9. The apparatus of claim 2, wherein the ADC comprises a switched capacitor digital to analog converter (DAC).

10. The apparatus of claim 2, wherein the reconfigurable filter and the ADC are fully differential.

11. A front end integrated circuit (IC) for biosignal acquisition, the front end IC comprising:
an analog front end comprising an instrumentation amplifier configured to receive a biosignal and a reconfigurable filter coupled to an output of the instrumentation amplifier, the instrumentation amplifier comprising a super class-AB output stage, the reconfigurable filter having a selectable gain and an adjustable bandwidth, the bandwidth adjusted based, at least in part, on a duty cycle of a clock signal;
an analog to digital converter (ADC); and
a duty cycle clock generator configured to provide the clock signal having the duty cycle to the reconfigurable filter and a second clock signal to the ADC.

12. The front end IC of claim 11, wherein the biosignal is selected from the group comprising electrocardiogram (ECG), electromyogram (EMG) and electroencephalogram (EEG).

13. The front end IC of claim 11, wherein the analog front end further comprises a programmable gain amplifier coupled between the instrumentation amplifier and the reconfigurable filter.

14. The front end IC according to claim 11, wherein the reconfigurable filter utilizes a switched-R-MOSFET-C (SRMC) topology.

15. The front end IC according to claim 11, wherein the instrumentation amplifier is a capacitively coupled chopper instrumentation amplifier.

16. The front end IC according to claim 11, wherein the ADC comprises a successive approximation register (SAR) configured to receive the second clock signal.

17. The front end IC according to claim 11, wherein the ADC comprises a switched capacitor digital to analog converter (DAC),

18. The front end IC according to claim 11, wherein the reconfigurable filter and the ADC are fully differential.

19. The front end IC according to claim 11, wherein the adjustable bandwidth is in the range of 100 Hertz (Hz) to 1 kilohertz (kHz).

20. The front end IC according to claim 11, wherein the adjustable bandwidth is in the range of 40 hertz (Hz) to 320 Hz with a 40 Hz step.
